(19) United States
(12) Patent Application Publication
(10) Pub. No.: US 2010/0004616 A1
(43) Pub. Date: Jan. 7, 2010

(54) FASTENING TAB AND SIDE PANEL OF DISPOSABLE DIAPER AND DISPOSABLE DIAPER

(75) Inventors: Tatsuya Nakamura, Tokyo (JP); Yasuhiro Kono, Kanagawa (JP); Takeshi Yamada, Kanagawa (JP); Jayshree Seth, Woodbury, MN (US)

Correspondence Address: 3M INNOVATIVE PROPERTIES COMPANY, PO BOX 33427, ST. PAUL, MN, 55133-3427, US

(73) Assignee: 3M INNOVATIVE PROPERTIES COMPANY, St. Paul, MN (US)

(21) Appl. No.: 11/721,097

(22) PCT Filed: Dec. 6, 2005
(86) PCT No.: PCT/US2005/044219

§ 371 Date: Sep. 23, 2009

(30) Foreign Application Priority Data
Dec. 7, 2004 (JP) 2004-354080

(51) Int. Cl. A61F 13/58

(52) U.S. Cl. 604/389

(57) ABSTRACT

There is provided a fastening tab or side panel for a disposable diaper (herein below sometimes referred to simply as a “fastening tab”) with an opening formed in the fastening tab or side panel base member or a notch on the terminal end of the fastening tab or side panel base member. When an invention fastening tab or side panel of an adult or baby disposable diaper (herein below sometimes referred to simply as a “diaper”) is pulled outward, the fastening tab or side panel in some embodiments can be pulled out by hooking the fastening tab or side panel at an opening (the “opening” formed by a discrete opening or a notch) with a finger. The opening helps the fastening tab to have good handleability on the diaper when the diaper is fixed to a user. A fastening tab having openings or notches, with the side having the notches being attached to the diaper such that the notch forms an “opening”, also distributes forces at the side of the diaper. When the fastening tab is pulled the resulting forces are distributed in at least two directions around the sides of the openings or notches. This improves the fit of the diaper around the waist and/or around the thighs. The opening(s) also improves the visibility of the location of the fastener attachment and increases comfort of the fastening tab.

The present invention relates to fastening tabs and side panels for use in disposable diapers, and the disposable diapers formed.

### BACKGROUND OF THE INVENTION

A disposable diaper generally is a laminated structure of a liquid-permeable top sheet which is brought into direct contact with a user, an absorber for absorbing and holding excrement, and a liquid-impermeable back sheet for inhibiting excrement from leaking. Disposable diapers are generally provided with some fastening system to securely fasten the diaper to a user or to adjust fastening conditions of the diaper.

The fastening system for a disposable diaper is generally constituted by the combination of a fixing member called a fastening tab or ear having a fastener which has a base member for the user to grasp for fixing the fastener of the fastening tab or ear. There has been proposed numerous fastening systems such as a tape fastening system using an adhesive tape as the fastener and mechanical fastening systems constituted by the combination of a loop and a hook.

With conventional diaper fastening systems the diaper is packed such that the fastening tabs are folded onto side panels of the diaper where the fastening tabs are further folded together with the side panels toward the inside of the diaper. Upon using the thus folded and packed diaper, the diaper is unfolded and the fastening tabs are pulled outward by grasping the fastening tab and then fixing the diaper to the user or baby. It is sometimes difficult to grasp the fastening tabs because the fastening tabs are adhered to the diaper when folded onto the diaper panel. Thus, the diaper does not have good handleability for a handler (note JP-B2-3222798 and JP-B2-3119443). Particularly, when a baby or infant is crying, the user or handler consciously wants to change the diaper as quickly as possible. This is a mental burden for the handler.

The present invention has been made in view of the problems of the conventional art and is characterized by providing a fastening tab, or a side panel of a disposable diaper, which can have good handleability, and/or comfort, and breathability and/or increased performance, and/or increasing effectiveness when the diaper is fastened to the user, and helping unfolding the folded diaper, or combinations of these properties.

### BRIEF SUMMARY OF THE INVENTION

With a fastening tab or side panel for a disposable diaper of the present invention (herein below sometimes referred to simply as a “fastening tab”) an opening or a notch is formed in the fastening tab or side panel base member. When an invention fastening tab or side panel of an adult or baby disposable diaper (herein below sometimes referred to simply as a “diaper”) is pulled outward, the fastening tab or side panel in some embodiments can be pulled by hooking the fastening tab or side panel at an opening (the “opening” formed by a discrete opening or a notch) with a finger. The opening helps the fastening tab to have good handleability on the diaper when the diaper is fixed to a user. A fastening tab having openings or notches, with the side having the notches being attached to the diaper such that the notch forms an “opening”, also distributes forces at the side of the diaper. When the fastening tab is pulled the resulting forces are distributed in at least two directions around the sides of the openings or notches. This improves the fit of the diaper around the waist and/or around the thighs. The opening(s) also improves the visibility of the location of the fastener attachment and increases comfort of the fastening tab.

### BRIEF DESCRIPTION OF THE DRAWINGS

Embodiments of the present invention will hereinbelow be described in detail with reference to the drawings. However, the present invention is by no means limited to the following embodiments, and it should be understood that the design may suitably be modified or improved on the basis of knowledge of those skilled in the art.

FIG. 1 is a plan view schematically showing an embodiment of a fastening tab for a disposable diaper of the present invention.

FIG. 2 is a plan view schematically showing another embodiment of a fastening tab for a disposable diaper of the present invention.

FIG. 3 is a plan view schematically showing still another embodiment of a fastening tab for a disposable diaper of the present invention.

FIG. 4 is a plan view schematically showing still another embodiment of a fastening tab for a disposable diaper of the present invention.

FIG. 5 is a plan view schematically showing still another embodiment of a fastening tab for a disposable diaper of the present invention.

FIG. 6 is a plan view schematically showing still another embodiment of a fastening tab for a disposable diaper of the present invention.

FIG. 7 is a plan view schematically showing still another embodiment of a fastening tab for a disposable diaper of the present invention.

FIG. 8 is a plan view schematically showing still another embodiment of a fastening tab for a disposable diaper of the present invention.

FIG. 9 is a plan view schematically showing still another embodiment of a fastening tab for a disposable diaper of the present invention.

FIG. 10 is a plan view schematically showing still another embodiment of a fastening tab for a disposable diaper of the present invention.

FIG. 11 is a plan view schematically showing still another embodiment of a fastening tab for a disposable diaper of the present invention.

FIG. 12 is a plan view schematically showing still another embodiment of a fastening tab for a disposable diaper of the present invention.

FIG. 13 is a plan view schematically showing still another embodiment of a fastening tab for a disposable diaper of the present invention.

FIG. 14 is a plan view schematically showing still another embodiment of a fastening tab for a disposable diaper of the present invention.

FIG. 15 is a plan view schematically showing still another embodiment of a fastening tab for a disposable diaper of the present invention.

FIG. 16 is a plan view schematically showing still another embodiment of a fastening tab for a disposable diaper of the present invention.

FIG. 17 is a plan view schematically showing still another embodiment of a fastening tab for a disposable diaper of the present invention.

FIG. 18 is a plan view schematically showing still another embodiment of a fastening tab for a disposable diaper of the present invention.

FIG. 19 is a plan view schematically showing still another embodiment of a fastening tab for a disposable diaper of the present invention.

FIG. 20 is a plan view schematically showing still another embodiment of a fastening tab for a disposable diaper of the present invention.

FIG. 21 is a plan view schematically showing still another embodiment of a fastening tab for a disposable diaper of the present invention.

FIG. 22 is a plan view schematically showing still another embodiment of a fastening tab for a disposable diaper of the present invention.

FIG. 23 is a plan view schematically showing still another embodiment of a fastening tab for a disposable diaper of the present invention.

FIG. 24 is a plan view schematically showing still another embodiment of a fastening tab for a disposable diaper of the present invention.

FIGS. 25(a) and 25(b) schematically show yet another embodiment of a fastening tab for a disposable diaper of the present invention. FIG. 25(a) is a plan view showing a notch in a base member, and FIG. 25(b) is a plan view showing an opening in the FIG. 25(a) base member.

FIG. 26 is a plan view schematically showing side panels of the present invention on a main body of a diaper.

FIG. 27 is a plan view schematically showing an embodiment of a disposable diaper of the present invention.

FIG. 28 is a plan view schematically showing another embodiment of a disposable diaper of the present invention.

FIG. 29 is a plan view schematically showing an example of a disposable diaper of the present invention.

FIG. 30 is a plan view schematically showing a comparative example of a disposable diaper.

FIG. 31 is a perspective view schematically showing an embodiment of a disposable diaper of the present invention.

FIG. 32 is a plan view schematically showing still another embodiment of a fastening tab for a disposable diaper of the present invention.

FIG. 33 is a plan view schematically showing still another embodiment of a fastening tab for a disposable diaper of the present invention.

FIG. 34 is a plan view schematically showing still another embodiment of a fastening tab for a disposable diaper of the present invention.

FIG. 35 is a plan view schematically showing still another embodiment of a fastening tab for a disposable diaper of the present invention.

FIG. 36 is a plan view schematically showing still another embodiment of a fastening tab for a disposable diaper of the present invention.

FIG. 37 is a plan view schematically showing still another embodiment of a fastening tab for a disposable diaper of the present invention.

FIG. 38 is a plan view schematically showing still another embodiment of a fastening tab for a disposable diaper of the present invention.

FIG. 39 is a plan view schematically showing still another embodiment of a fastening tab for a disposable diaper of the present invention.

FIG. 40 is a plan view schematically showing still another embodiment of a fastening tab for a disposable diaper of the present invention.

FIG. 41 is a plan view schematically showing still another embodiment of a fastening tab for a disposable diaper of the present invention.

FIG. 42 is a plan view schematically showing still another embodiment of a fastening tab for a disposable diaper of the present invention.

FIG. 43 is a plan view schematically showing still another embodiment of a fastening tab for a disposable diaper of the present invention.

FIG. 44 is a plan view schematically showing an embodiment of side panel using a fastening tab for a disposable diaper of the present invention.

FIG. 45 is a perspective view schematically showing an embodiment of a disposable diaper of the present invention.

FIG. 46A-D are a plan views schematically showing embodiments of side panels having a fastening tab for a disposable diaper of the present invention, cut from a continuous roll.

### DESCRIPTION OF EMBODIMENTS OF THE INVENTION

The invention is directed at a fastening tab for a disposable diaper (open or pant-style) side panel or a side panel used as or with a fastening tab for a disposable diaper for a baby or an adult Each can give a disposable diaper excellent fit, comfort and visibility.

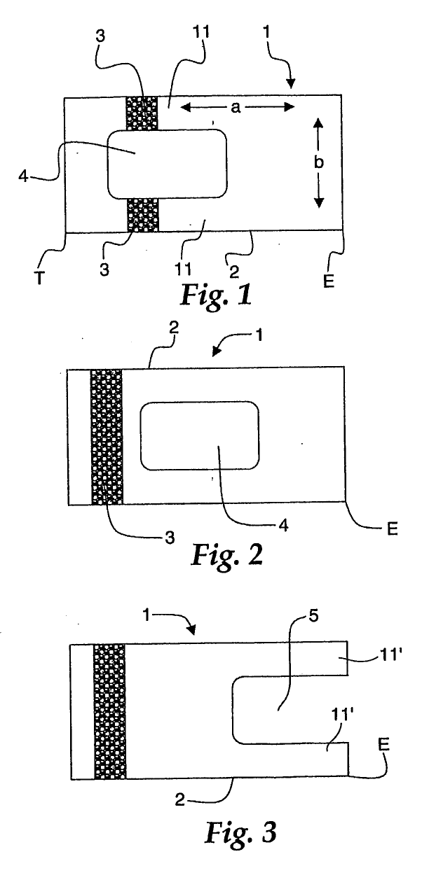

FIG. 1 is a plan view schematically showing a first embodiment of an invention fastening tab for a disposable diaper.

As shown in FIG. 1, a fastening tab 1 of the present embodiment has a rectangular base member 2, and a fastener 3 disposed on a surface of the base member. A hole-shaped opening 4 where the tab may be hooked with a finger is formed in the base member 2. In the fastening tab 1 shown in FIG. 1, the direction a shown by an arrow is defined as a longitudinal direction a, and the direction b shown by an arrow is a lateral direction b. The fastening tab 1 shown in FIG. 1 is attached to a diaper at an attached end portion E, in the longitudinal direction a of the base member.

With the opening 4 the tab can be hooked with a finger. This allows the fastening tab 1 to be easily pulled with a finger at the opening 4, thus improving the fastening tab handleability.

When the tab 1 is pulled with a finger at the opening 4, the tab 1 is pulled toward the terminal end portion T opposite to the attached end portion E at the diaper side edge. Since an opening 4 is formed in the base member 2, two belt-like portions 11 extending in the longitudinal direction a are formed in parallel, with the opening 4 interposed therebetween. Therefore, when the fastening tab is applied to a diaper, the two belt-like portions 11 are placed under tension. Therefore, one belt-like portion 11 which is closer to the user's waist applies force around the user's waist to improve fit around the user's waist, and the other belt-like portion 11 which is closer to the user's thigh applies force around the user's thigh to improve fit around the user's thigh. This allows the forces applied by the fastening tab when under tension to be directed by the belt-like portions 11.

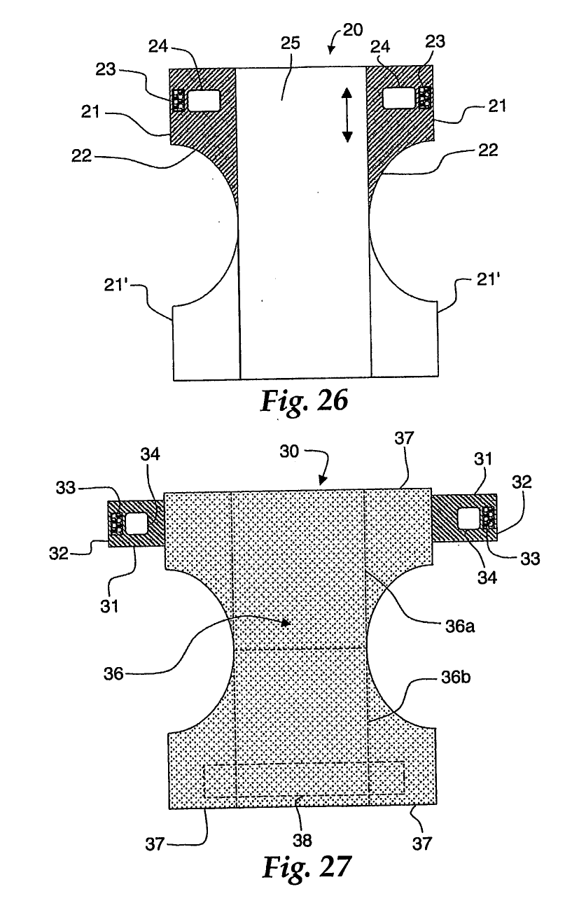

On the landing zone 38 as shown in FIG. 27, where a fastening tab is engaged, can be printed a design such as a character, a number showing a position of engagement, a vertical line, or the like. When a conventional fastening tab is engaged on the landing zone, the fastening tab covers the print. However with a fastening tab of the present embodiment provided with openings, the print is partially visible. This permits more accurate placement of the fastening tab on the landing zone.

With fastening tabs of the present embodiment, it is preferable that at least a part of the diaper side panel including the fastening tab or the fastening tab is formed out of elastic material. Generally, at least a part of the side panel or fastening tab is formed out of elastic material. When the diaper is attached to a user's body with the fastening tab, the elastic in the side panel or fastening tab presses against the user's body with a moderate force keeping the diaper fit around the user's body. As an elastic material, elastomeric films, elastomer fabrics, laminates thereof, or composites thereof can be employed (see, for example, JP-B-6-94188, U.S. Pat. Nos. 5,691,034, and 5,501,679). It is also possible to use elastic materials singly or in combination. Examples of suitable elastic polymers to form the elastic materials include natural rubber, isoprene rubber, 1,2-polybutadiene, styrene-butadiene rubber, chloroprene rubber, nitrile rubber, butyl rubber, ethylene-propylene rubber, or urethane rubber. Examples of the elastic fabrics include various fibrous materials such as woven fabrics and nonwoven fabrics. Examples of nonelastic fibers useful in fibrous laminates or composites include synthetic fibers, such as fibers of polyethylene, polypropylene, polyester, and polyamide and natural fibers, such as fibers of cotton and rayon. The thickness of the elastic material can be in a wide range and is preferably about 50 to 1000 μm.

It is not necessary for the base member of a fastening tab of the present invention to have a rectangular shape as shown in FIG. 1, and it may have any suitable shape, as will be discussed with other specific embodiments.

In a fastening tab of the present invention, there is no limitation to the size of the base member, and the size can be suitably determined in accordance with the size of a diaper to be used. For example, the size of a fastening tab for an adult diaper can be (lateral direction×longitudinal direction) 625 mm² (e.g. 25 mm×25 mm) to 1400 mm² (120 mm×120 mm), and the size of a fastening tab for a baby diaper can be 225 mm² (e.g. 15 mm×15 mm) to 10000 mm² (100 mm×100 mm). It is preferable that any belt-like portions have a width of 5 mm or more.

Materials for forming the base member are preferably synthetic thermoplastic resins such as polypropylene, polyethylene, poly(ethylene terephthalate) and nylon. In some cases the base member can employ a woven or nonwoven fabric constituted by various fibers, paper, or the like. If the base member of a fastening tab of the present embodiment is very soft, a reinforcing member 13 may be used (see, e.g., FIG. 11). As the reinforcing member, a plastic film, a nonwoven fabric, a woven fabric, or the like, can be used, which is more rigid than the soft base member.

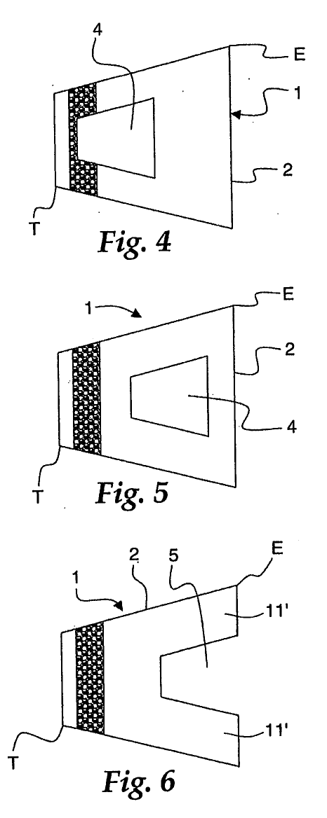

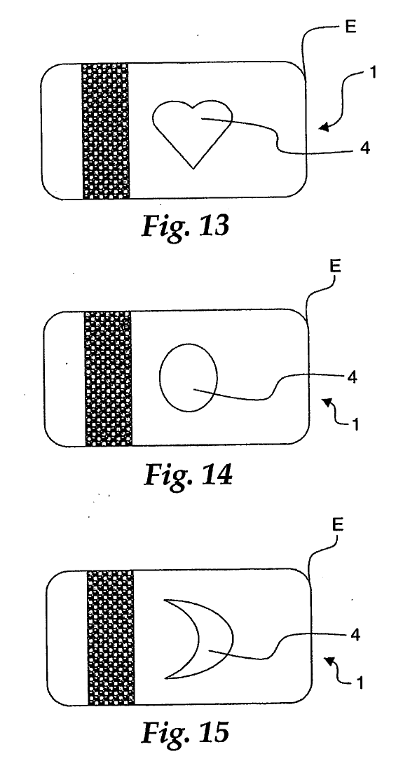

In a fastening tab of the present invention, the shape of an opening is not particularly limited. The shape may be triangular, rectangular (see FIG. 1), square, trapezoidal (see FIG. 4), circular, elliptical (see FIG. 14), race-track-shaped, heart-shaped (see FIG. 13), or crescent (see FIG. 15). These exemplary shapes generally have at least one apex thereof being rounded in a circular arc. For example, the opening 4 formed in the fastening tab 1 shown in FIG. 1 is rectangular, and the four apexes are formed to give a shape of a circular arc. There is no particular limitation to the size of the opening, and the size may be determined according to the size of a diaper to be used and the desired function of the opening or openings. For example, the opening size for an adult diaper having a generally square opening can be (lateral direction x longitudinal direction) 400 mm² (e.g. 20 mm×20 mm) to 10000 mm² (e.g. 100 mm×100 mm), and the opening size for a baby diaper can be 100 mm (e.g. 10 mm×10 mm) to 6400 mm² (e.g. 80 mm×80 mm), if it is desired to provide an opening that can be grasped by a finger. Smaller openings used solely for increased flexibility, for creating belt-like extensions for force distribution or the like are also envisioned, particularly smaller openings formed by notches at the attached end portion E of the fastening tab.

As a fastener, there may be used a mechanical fastener, an adhesive tape, or the like. Mechanical fasteners are preferable. Since a mechanical fastener is fastened with a combination of a hook having a number of micro-protrusions or hooking elements, and a loop to engage with the hook, either the hook or the loop may form the fastener on the fastening tab with the corresponding element forming the landing zone. Conventional mechanical fasteners can be used. In the case of the hook, the shape of hooking micro-protrusions may be T-shaped, mushroom-shaped, hook-shaped, or the like. Materials suitable for forming the hook could be thermoplastic polymers such as polyethylene, polypropylene, polyester, nylon, or the like. The loop could be formed, for example, from woven fabrics, nonwoven fabric, or the like. Material for forming a loop fiber could likewise be thermoplastic polymers such as polypropylene, polyethylene, polyester, nylon, or the like.

The size of the fastener can suitably be determined in accordance with the size of the fastening tab or side panel. For example, the size of the fastener (lateral direction×longitudinal direction) could be 25 mm² (e.g. 5 mm×5 mm) to 600 mm² (e.g. 120 mm×50 mm) for an adult diaper, and 25 mm² (e.g. 5 mm×5 mm) to 4000 mm² (e.g. 100 mm×40 mm) for a baby diaper. When a fastening tab of the present embodiment is attached to a diaper, it is attached so that a surface of the fastening tab having the fastener faces inside (the side where the fastening tab is brought in contact with a user) when the diaper is applied to the user.

FIG. 2 is a plan view schematically showing another embodiment of a fastening tab of the present invention. As shown in FIG. 2, a fastening tab 1 may be formed in such a manner that the opening 4 is not brought in contact (does not overlap) with the fastener 3. The distance between the opening 4 and the fastener 3 on the base member can be 5 mm or more. FIG. 3 is a plan view schematically showing another embodiment of a fastening tab of the present invention. As shown in FIG. 3, an opening 5 is formed by a notch on the one attached end portion E of the base member 2 to be attached to the diaper side edge. The notch opening 5 is formed by the attached end portion E when on the diaper side edge where a central portion of the notch forms the opening 5 and with end portions of the belt-like extensions 11′ in the lateral direction attached to the diaper side edge. As shown, the notch opening 5 is formed by the fastening tab 1 and the diaper to have a structure similar to that of a fastening tab 1 opening 4 in FIG. 1, which has a similar effect when the fastening tab 1 is disposed on the diaper or diaper side panel. The use of a notch opening on the fastening tab has the additional benefit of reducing the stiffness of the attached end portion E when attached to the diaper or diaper side panel. This is important in increasing comfort for the user by reducing stiffness in this area and potential red marking of the user's skin. The belt-like extensions 11′ can also be rounded to decrease sharp corners where attached to the diaper.

FIGS. 4 to 6 are plan views schematically showing still other embodiments of fastening tabs of the present invention. In each fastening tab 1 shown in FIGS. 4 and 5, an opening 4 is formed in a trapezoidal base member 2. In the fastening tab 1 shown in FIG. 6, an opening 5 is formed by the notch on the attached end portion E of trapezoidal base member 2 when attached to the diaper. In each fastening tab 1 shown in FIGS. 4 to 6, the fastener 3 is disposed near the terminal end portion T in such a manner that a tip of the base member 2 remains exposed.

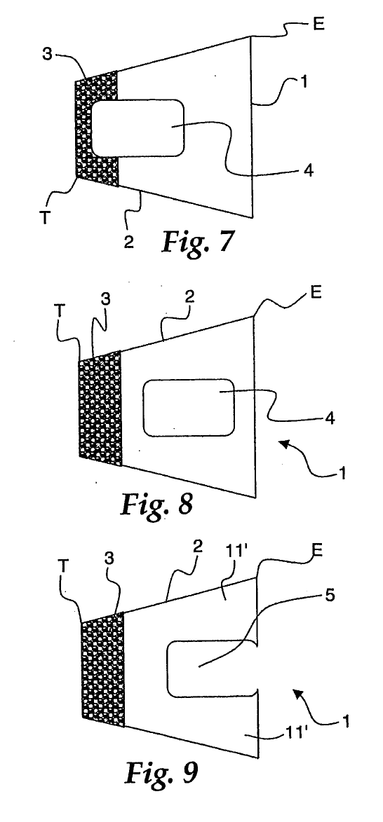

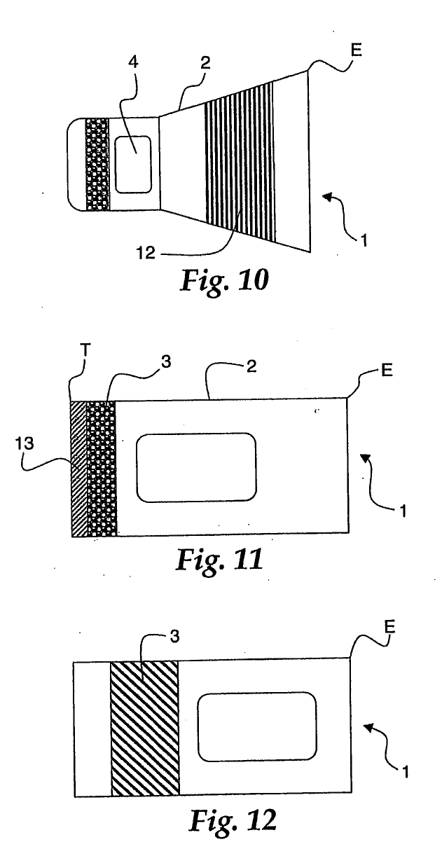

FIGS. 7 to 9 are plan views schematically showing still other embodiments of fastening tabs of the present invention. In each fastening tab 1 shown in FIGS. 7 and 8, a rectangular opening 4 is formed in a trapezoidal base member 2. In the fastening tab 1 shown in FIG. 9, a rectangular opening 5 is formed in a trapezoidal base member 2. In each fastening tab 1 shown in FIGS. 7 to 9, the fastener 3 extends to the terminal end portion T in such a manner that a tip of the base member 2 is covered by the fastener 3 (the fastener 3 forms the tip of the terminal end portion T).

FIG. 10 is a plan view schematically showing still another embodiment of a fastening tab of the present invention. In the fastening tab 1 shown in FIG. 10, the base member 2 has portion with a rectangular shape connected to a parallel side of a trapezoidal shaped portion, and an opening 4 is formed in a rectangular portion of the base member 2. Further, an elastic portion 12 is provided on the trapezoidal portion. By thus forming an elastic portion 12 on at least a portion of the fastening tab 1, fit of the diaper, when the fastening tab is applied to a user, is improved.

FIG. 11 is a plan view schematically showing still another embodiment of a fastening tab of the present invention. In the fastening tab 1 shown in FIG. 11, a reinforcing member 13 (such as a film) is disposed on the terminal end portion T, and the fastener 3 is disposed adjacently to the reinforcing member 13. By thus disposing a reinforcing member 13 on the terminal end portion T of a fastening tab 1, the fastening tab can be more easily handled if the base member 2 is soft, also improving handleability. Materials suitable for forming a reinforcing film member 13 include, polypropylene, polyethylene, poly(ethylene terephthalate), nylon, or the like as discussed above.

FIG. 12 is a plan view schematically showing still another embodiment of a fastening tab of the present invention. In the fastening tab 1 shown in FIG. 12, the fastener 3 is an adhesive tape portion. Incidentally, in each embodiment of a fastening tab of the present invention shown in the Figures other than the embodiment shown in FIG. 12, the fastener 3 is preferably a mechanical fastener.

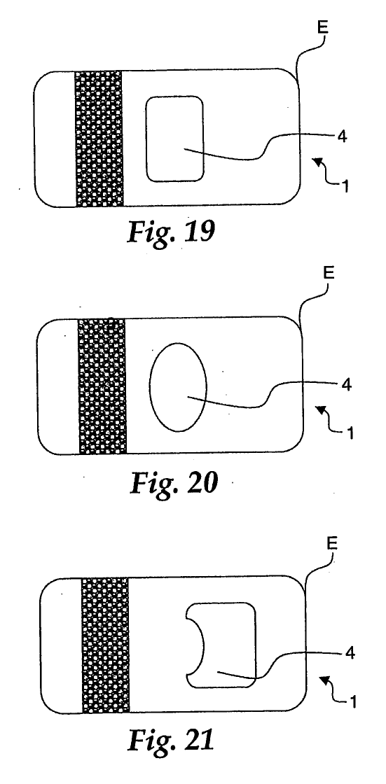

FIGS. 13 to 15 and 19 to 21 show plan views schematically showing still other embodiments of fastening tabs of the present invention. In these embodiments, the openings 4 are varied in their shapes. The fastening tab 1 shown in FIG. 13 has a heart-shaped opening 4, the fastening tab 1 shown in FIG. 14 has an elliptical opening 4, the fastening tab 1 shown in FIG. 15 has a crescent opening 4, the fastening tab 1 shown in FIG. 19 has a quadrangular opening 4, the fastening tab 1 shown in FIG. 20 has an elliptical opening 4 whose longer diameter is about twice as long as the shorter diameter, and the fastening tab 1 shown in FIG. 21 has an opening having a shape of a rectangle with one side protruding in a circular arc state. Each of the shapes may be oriented in any direction.

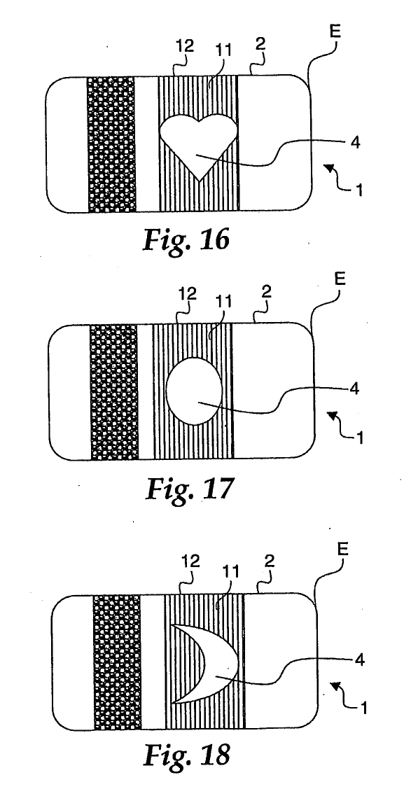

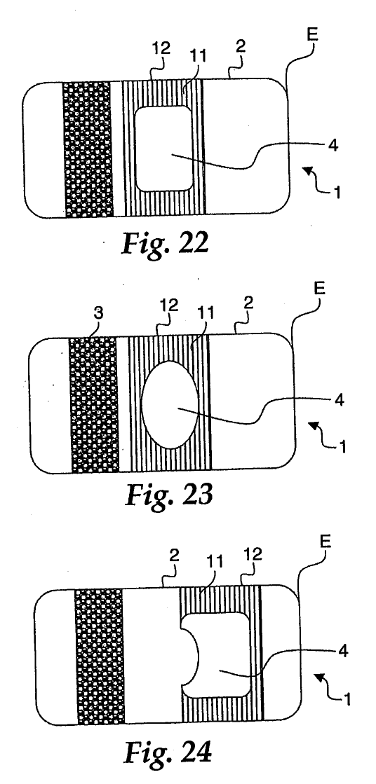

FIGS. 16 to 18 and 22 to 24 are plan views schematically showing still other embodiments of a fastening tab 1 of the present invention. Each of these has an elastic portion 12 in apart of the base member 2. The elastic portion 12 is formed between both end portions in a lateral direction, and an opening 4 is formed in the elastic portion 12. As shapes of the openings 4, fastening tabs 1 shown in FIGS. 16 to 18 are the same as those of the fastening tabs 1 shown in FIGS. 13 to 15, and fastening tabs 1 shown in FIGS. 22 to 24 are the same as those of the fastening tabs 1 shown in FIGS. 19 to 21. This has the advantage of reducing the amount of elastic material used on the fastening tab while increasing the functionality of the elastic used by concentrating the stress in the belt-like portions 11.

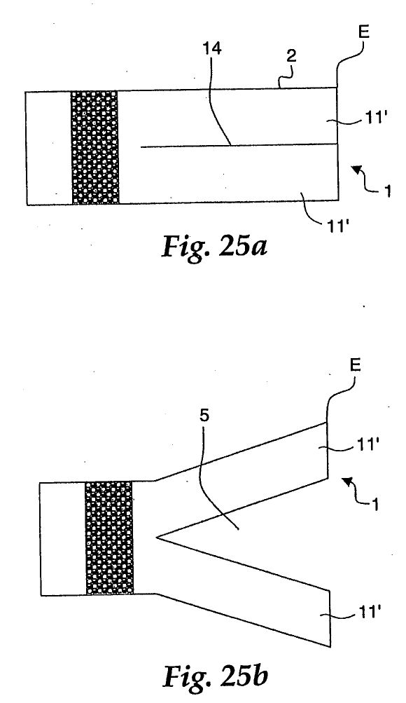

FIGS. 25(a) and 25(b) schematically show still other embodiments of fastening tabs of the present invention. FIG. 25(a) is a plan view showing a notch 14 formed in the base member, and FIG. 25(b) is a plan view showing an opening formed in the base member. The notch 14 is capable of forming an opening 5 in the base member as shown in FIG. 25(b). That is, by opening the notch 14, an opening 5 is formed as shown in FIG. 25(b). In this case, the opening 5 is triangular. The fastening tab 1 of the present embodiment is disposed on the diaper in the state that the notch is opened to form an opening 5 as shown in FIG. 25(b) with belt like portions 11′.

FIG. 26 is a plan view schematically showing a main body 25 of a disposable diaper 20 of the invention having a side panel. A side panel 21 of the present embodiment has a side panel base member 22 and a fastener 23 disposed on a surface of the panel base member 22. An opening 24 is formed on the panel base member 22. At the opening 24 formed in the panel base member 22, the panel base member 22 can be hooked with a finger. The opening 24 may be formed by an attached end portion of the panel base member 22 (for example, it may be formed as the opening 5 in FIG. 3). One end portion of the panel base member 22 is an attached end portion E on the side where the side panel is attached to the main body 25 of a disposable diaper 20. In this case, the panel base member 22 is attached to a diaper with the notch forming an opening. A side panel 21 of the present embodiment shown in FIG. 26 is disposed on a portion corresponding to the back panel of the main body 25 of a disposable diaper. In the main body 25 for a disposable diaper, an ordinary side panel 21′ is further disposed on a portion corresponding to the front panel. The side panels of the present embodiment may have a shape to allow unitarily joining of the side panel 21 and the ordinary side panel 21′ in FIG. 26.

An opening formed in a side panel of the present embodiment can have the same shape as an opening formed in the aforementioned fastening tabs of the present invention, for example, triangular, rectangular, square, trapezoidal, elliptical, race-track-shaped, heart-shaped, or crescent. An opening portion can have a length of 10 to 300 mm in a lateral direction (lateral direction b) of the side panel 21.

In a base member constituting a side panel of the present embodiment, it is preferable that at least apart of the side panel base member is formed of an elastic material. If the base member is formed at least in part of an elastic material, then when the diaper is applied to a user's body with a fastener, the panel member increases fit and comfort. It is preferable that the elastic material materials and thickness can be similar to those of elastic materials used with fastening tab base members of the present invention. A base member material and shape for a side panel of the present embodiment are not particularly limited and may suitably be determined in accordance with a diaper to be produced. In addition, the size of the side panel base member can suitably be determined in accordance with a size of a diaper. In the case of an adult diaper, the size can be 5000 mm² (e.g. 100 mm×50 mm) to 80000 mm² (e.g. 400 mm×200 mm), and for a baby diaper, the size can be 1000 mm² (e.g. 50 mm×20 mm) to 20000 mm² (e.g. 200 mm×100 mm).

For a side panel of the present embodiment the fastener is preferably a mechanical fastener or an adhesive tape, and a mechanical faster is particularly preferable. The fastener 5 material is preferably similar to that of the fastener material used on the fastening tabs of the present invention. The fastener may be disposed on a side panel base member along the terminal end portion of the side panel, and the fastener may be disposed on a side panel near the terminal end portion thereof with the panel base member remaining exposed on the terminal end portion. The fastener material could also be placed on a separate fastening tab, which could be a conventional fastening tab or a fastening tab in accordance with the invention.

A side panel of the present embodiment can be produced in almost the same method as the method for producing a side panel where an ordinary mechanical fastener or an adhesive tape is disposed as a fastener. The difference is the formation of openings having a predetermined shape or notches

An embodiment of a disposable diaper of the present invention will be described. In a disposable diaper of the present embodiment, the aforementioned fastening tab or side panel of the present invention is disposed on a general use disposable diaper.

FIG. 27 is a plan view schematically showing an embodiment of a disposable diaper of the present invention. A disposable diaper 30 of the present embodiment is provided with the main body 36 for a disposable diaper and side panels 37 for a disposable diaper, the side panels being disposed on the main body 36 for a disposable diaper. Further, the aforementioned fastening tabs 31 for a disposable diaper are disposed on side panels 37.

The disposable diaper 30 has a main body 36 having a laminated structure of a liquid-permeable top sheet which is brought into direct contact with a user, an absorber for absorbing and holding excrement, and a liquid-impermeable back sheet for inhibiting excrement from leaking. The disposable diaper 30 is generally provided with the fastening tab 31 of the present invention to securely fix the disposable diaper 30 to a user or to adjust the fastening conditions of the disposable diaper 30.

As shown in FIG. 27, the main body 36 of a disposable diaper of the present embodiment is constituted by a front end panel 36b for covering a user on the abdomen side and a back end panel 36a for covering a user on the buttocks side. A side panel 37 is disposed on each side portion (each side) of each of the front panel 36b and the back panel 36a. On each of the side panels 37 of the back panel 36a is disposed a fastening tab 31. In the vicinity of the upper end of the front panel 36b (on the side where the diaper is not brought in contact with a user) is disposed a landing zone 38 where the fastening tabs 31 adhere. In the base member 32 of the fastening tab 31 is formed an opening 34, and a fastener 33 (hook-type mechanical fastener) is disposed on the base member 32. The side panels 37 are not necessarily separated into two distinct pieces. On the front panel 36b side and on the back panel 36a side they may have a unitarily joined structure such as being formed of one integral material.

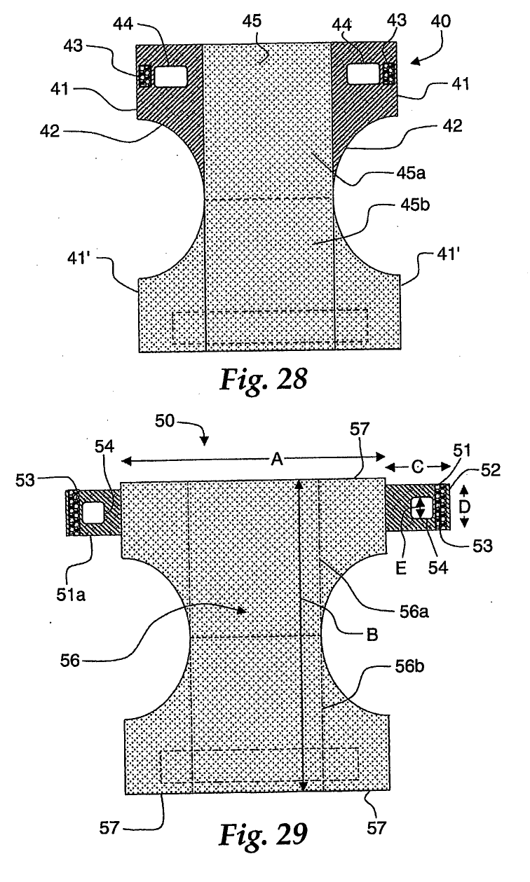

FIG. 28 is a plan view schematically showing another embodiment of a disposable diaper of the present invention. The disposable diaper 40 of the present embodiment is constituted by a main body 45 and the aforementioned side panels 41 of the present invention disposed on one end panel 45a of the main body 45. A side panel 41 has a panel base member 42 and a fastener disposed on a surface of the panel base member 42, and an opening 44 is formed in the panel base member 42. The main body 45 is constituted by a back end panel 45a and a front end panel 45b. The side panels 41 of the present invention are disposed on the back panel 45a, and a conventional side panel 41′ is disposed on the front panel 45b.

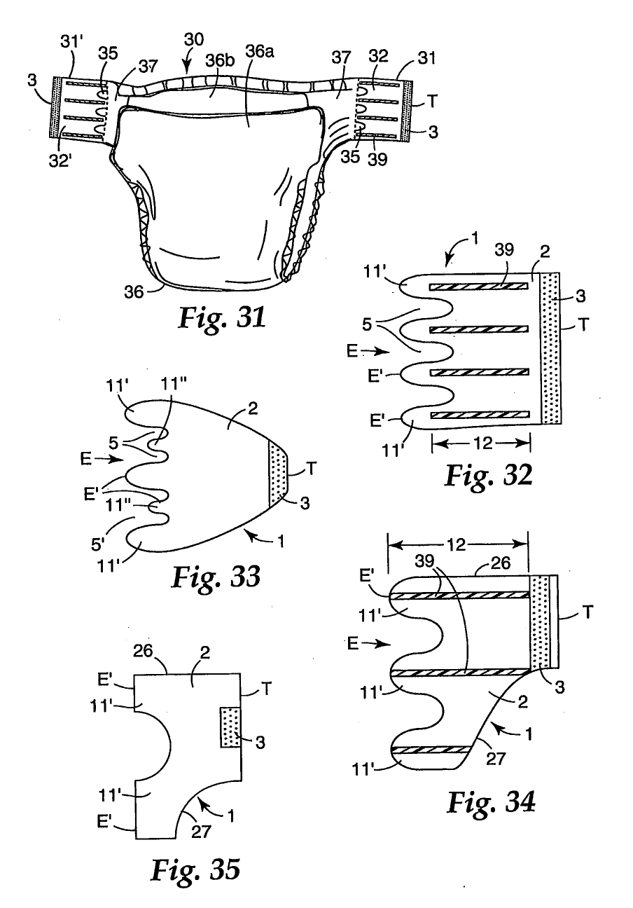

FIG. 31 is a perspective view of another embodiment of a disposable diaper 30 using another embodiment of an invention fastening tab 31 having multiple notches forming multiple openings 35 placed on the side panels 37 of the disposable diaper 30 discussed in reference to FIG. 27. The details of this fastening tab of the present invention are shown in reference to FIG. 32. The fastening tab 1 has a rectangular base member with a terminal end portion T provided with a fastener 3. The fastening tab 1 of this embodiment of the present invention is attached to a diaper at the attached end portions E′ of the belt-like portions 11′. This produces a number of openings 5 (35 on the diaper of FIG. 31) each opening associated with two belt-like portions 11′. The belt-like portions 11′ are each aligned with discrete elastic members 39. The discrete elastic members are shown as parallel but could extend in any suitable direction where elastic forces are desired. This elastic portion 12 of the base member 2, formed by portions of the discrete elastic portions, would be made stretchable by conventional methods such as incremental stretching of the base member 2, corrugation of any inelastic layer forming the base member 2 or like techniques. Generally, the base member 2 in this embodiment would be a laminate of an inelastic material such as a film or nonwoven onto one or both faces of the elastic members 39. Preferably, the base member would be a laminate of two relatively inelastic materials or either side of the elastic members 39, preferably fibrous webs such as nonwoven fibrous webs of synthetic thermoplastic polymers, such as spunbond webs, meltblown webs, spunlace web, carded webs or the like.

The belt-like portions 11′ direct the forces of the discrete elastic elements onto the side of the diaper more fully utilizing the elastic while distributing the tension forces as desired. These belt-like portions 11′ also soften the attached end portions E′ when attached to the diaper by reducing the rigidity of the connection of the fastening tab on the diaper. Use of multiple fastening tab belt-like portions 11′ also creates tear stops preventing tear propagation at the attached end portion E.

FIG. 33 is a plan view schematically showing another embodiment of a fastening tab 1 of the present invention similar to FIG. 32, openings 5 are formed by multiple notches on the attached end portion E of the base member 2. The notch openings 5 are formed by two sets of belt-like extensions 11′ and 11″ extending different degrees toward the attached end portions E′. The belt-like extensions 11″ terminate short of the belt-like extensions 11′, which can help in reducing material usage but still, provide for tear stops and increased flexibility. These belt-like extensions 11′ and 11″ could also be associated with discrete elastic members to create an elastic portion, not shown.

FIG. 34 is a plan view schematically showing another embodiment of a fastening tab of the present invention formed in the shape of a side panel. The terminal end portion T is narrower than the attached end portion E′ of the base member 2. The base member has a flat top edge 26 and a shaped bottom edge 27, which forms a portion of the leg engaging opening on the diaper. The discrete elastic members 39 are aligned with the belt-like portions 11′ to provide elastic fit for the fastening tab. The elastic portion 12 is shown as extending the full length of the discrete elastic members 39, however, the elastic portion 12 could only comprise a portion of the length of the discrete elastic members 39 by selectively providing the base portion to be extensible in a select portion.

FIG. 35 is a plan view schematically showing another embodiment of a fastening tab of the present invention formed in the shape of a side panel. The terminal end portion T again is narrower than the attached end portion E′ of the base member 2. The base member has a flat top edge 26 and a shaped bottom edge 27, which forms a portion of the leg engaging opening on the diaper. Two belt-like portions 11′ are used to provide fit for the fastening tab.

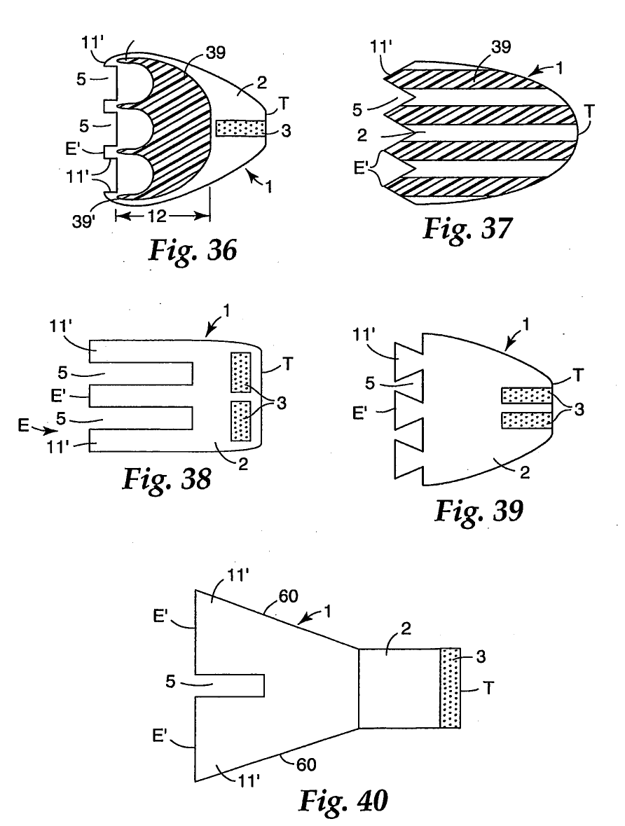

FIG. 36 is a plan view schematically showing another embodiment of a fastening tab of the present invention. The fastening tab 1 has a symmetrically curved base member 2 with the terminal end portion T provided with a fastener 3. The fastening tab 1 of this embodiment of the present invention is attached to a diaper at the attached end portions E′ of the belt-like portions 11′. This produces a number of openings 5 each associated with two belt-like portions 11′. The belt-like portions 11′ are aligned with a single discrete elastic member 39, having extensions 39′ that extend toward the belt-like portions 11′ such that the belt-like portions 11′ direct the forces of the discrete elastic elements onto the side of the diaper. The smaller openings in this embodiment, and like embodiments would not provide an opening to increase handleability or visibility but would provide a soft attachment to the diaper and breathability as well as tear stops.

FIG. 37 is a plan view schematically showing another embodiment of a fastening tab 1 of the present invention. The fastening tab 1 like in FIG. 36 has a symmetrically curved base member 2, however the terminal end portion T is not provided with a fastener 3. The fastening tab 1 of this embodiment of the present invention is attached to a diaper at the attached end portions E′ of the belt-like portions 11′. This produces a number of openings 5 each associated with two belt-like portions 11′. The belt-like portions 11′ are each aligned with discrete elastic members 39, that extend into the belt-like portions 11′ such that the belt-like portions 11′ direct the forces of the discrete elastic elements onto the side of the diaper.

FIG. 38 is a plan view schematically showing another embodiment of a fastening tab of the present invention. The terminal end portion T again is generally the same width as the attached end portion E of the base member 2. The belt-like portions 11′ are used to provide fit for the fastening tab and extend for a length along the tab to create larger openings 5.

FIG. 39 is a plan view schematically showing another embodiment of a fastening tab 1 of the present invention. The fastening tab 1 like in FIG. 36 has a symmetrically curved base member 2 with the terminal end portion T provided with two fasteners 3. The fastening tab 1 of this embodiment of the present invention is attached to a diaper at the attached end portions E′ of the belt-like portions 11′. This produces a number of openings 5 each associated with two belt-like portions 11′. The belt-like portions 11′ taper outwardly at both sides as they extend to the attached end portions 11′ which allows for wider openings 5.

FIG. 40 is a plan view schematically showing another embodiment of a fastening tab 1 of the present invention. The fastening tab 1 has a symmetrically tapered base member 2 with the terminal end portion T provided with a fastener 3. The fastening tab 1 of this embodiment of the present invention is attached to a diaper at the attached end portions E′ of the two belt-like portions 11′. This produces one opening 5 associated with the two belt-like portions 11′. The belt-like portions 11′ taper outwardly on the outer sides 60 only as they extend to the attached end portions 11′ which allows for a rectangular opening 5 and wider attached end portions E′.

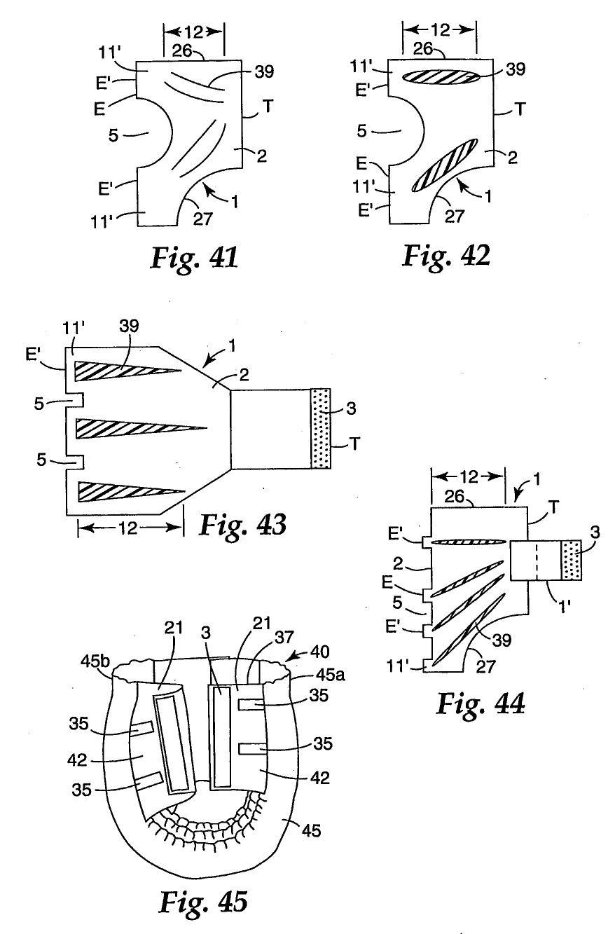

FIGS. 41 and 42 are plan views schematically showing other embodiments of a fastening tab of the present invention formed in the shape of a side panel, similar to that shown in FIG. 35. The terminal end portion T again is narrower than the attached end portion E of the base member 2. The base member has a flat top edge 26 and a shaped bottom edge 27, which forms a portion of the leg engaging opening on the diaper. Two belt-like portions 11′ are used to provide fit for the fastening tab. Discrete elastic members 39 are placed in the base member 2 generally aligned with the belt like portions 11′.

FIG. 43 is a plan view schematically showing another embodiment of a fastening tab I of the present invention. The fastening tab 1 has a symmetrically tapered base member 2 with the terminal end portion T provided with a fastener 3. The fastening tab 1 of this embodiment of the present invention is attached to a diaper at the attached end portions E′ of the belt-like portions 11′. This embodiment produces two openings 5 associated with the three belt-like portions 11′. The belt-like portions 11′ each are associated with discrete elastic members 39 which direct the elastic force directly toward the belt like portions 11′.

FIG. 44 is a plan view schematically showing another embodiment of a fastening tab of the present invention formed in the shape of a side panel, similar to that shown in FIG. 35 but having an attached end portion E similar to that described in FIG. 36. The terminal end portion T again is narrower than the attached end portion E of the base member 2. The base member has a flat top edge 26 and a shaped bottom edge 27, which forms a portion of the leg engaging opening on the diaper. The belt-like portions 11′ are used to provide fit for the fastening tab. Discrete elastic members 39 are placed in the base member 2 generally aligned with the belt like portions 11′. The fastener 3 is provided by an attached conventional fastening tab 1′.

FIG. 45 is a plan view schematically showing another embodiment of a disposable diaper of the present invention. This diaper is a pant style diaper where the two side panels of the invention 21 are removably engagable such as by a mechanical fastener 3 or cohesive adhesives or like removably engagable fasteners. The disposable diaper 40 of the present embodiment is constituted by a main body 45 and the aforementioned side panels 21 of the present invention disposed on one or both sides of both end panels 45a and 45b of the main body 45. A side panel 21 has a panel base member 42 and a fastener 3 disposed on a surface of the panel base member 42, and openings 35 are formed in the panel base member 42.

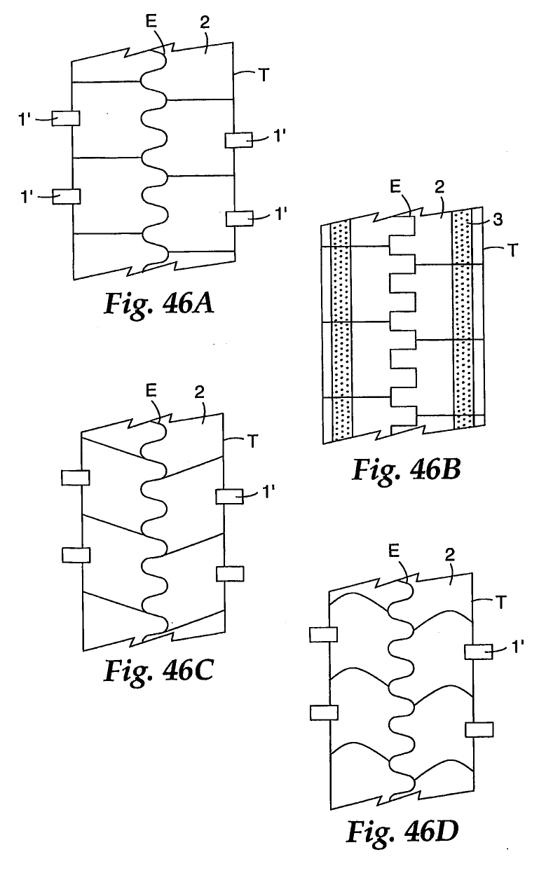

FIGS. 46A-46D show ways to form fastening tab of the present invention from a continuous roll of fastening tab base material 2. As shown, the notches are formed by a sinusoidal cut eliminating waste and forming mirror image attached ends E with belt-like portions and notches. The fastener can be integral with the base material 2 as shown in FIG. 46b or provided on an attached conventional fastening tab.

It should be understood that all the features described in detail with respect to any specific embodiment could be used with the other embodiments of the invention as would be apparent to one of skill in the art. Also although the terms side panel and fastening tab are both used the term fastening tab is meant to include side panels having the invention features and either an integral or attached fastener (e.g. by a conventional fastening tab). It is also possible that side panels of the invention are provided without an integral or attached fasteners which could be used for example with a pant style diaper where the terminal ends of two opposing side panels on opposite end panels are permanently joined by means of ultrasonic welding, heat bonding, adhesives or combinations of joining methods.

The present invention will hereinbelow be described more specifically with Examples. However, the present invention is by no means limited to these Examples.

A disposable diaper having a structure shown in FIG. 29 was produced. Side panels 57 were disposed on both sides of a main body 56 constituted by a front panel 56b and a back panel 56a. A fastening tab 51 having a rectangular base member 52 provided with a hook mechanical faster (fastener) 53 and a rectangular opening 54 was disposed on the back panel 56a side in a known manner. Two kinds of diapers, adult and baby, were produced.

For the main bodies for the diapers and the side panels, a large-sized Pampers “Cotton Care”™ was used for a baby diaper, a medium-sized “Attends”™ was used for an adult diaper.

The baby diaper had a main body 56 longitudinal length B of 500 mm, a transverse length A with the unfolded side panels of 320 mm, a length C of a fastening tab 51 of 60 mm, and a fastening tab 51 width D of 60 mm. There were produced three kinds (Examples 1 to 3) of baby diapers where the openings 54 have varied widths E as shown in Table 1.

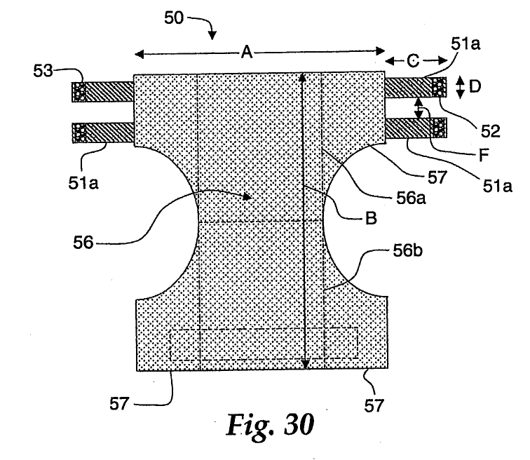

The diaper for an adult had a main body longitudinal length B 56 of 850 mm, a transverse length A with the unfolded side panels of 650 mm, a length C of a fastening tab 51 of 80 mm, and a width D of the fastening tab 51 of 160 mm. There were produced two kinds (Examples 4 to 5) of adult briefs where the openings 54 have varied widths E as shown in Table 1. In an adult brief, two belt-shaped fastening tabs were disposed at an interval shown in FIG. 30 (Comparative Example 1). In FIG. 30, the same reference numerals as in FIG. 29 denote the same elements as in FIG. 29. The fastening tab 51a had a length C of 80 mm and a width (width for one belt) D of 30 mm. The interval F between two belts was 100 mm.

The briefs were tested for “easiness in peeling” and “easiness in fastening”.

The “easiness in peeling,” was evaluated by a handler's sense in peeling a fastening tab which was folded in a manner such that a fastener stuck to a side panel. The results are shown in Table 1. In Table 1, “good” means that it could easily be peeled, “fair” means that there was no problem for practical use though peeling was difficult, and “bad” means that peeling was difficult.

The “easiness in fastening” was evaluated by a handler's sense in fixing a fastening tab to the front panel (landing zone) when the diaper is fixed to a user. In Table 1, “good” means that it can easily be fixed, “fair” means that there was no problem for practical use, though fixing was difficult, and “bad” means that fixing was difficult.

TABLE 1

| | Width E of | Easiness in | Easiness in |
|---|---|---|---|
| | opening (mm) | peeling | fastening |
| Example 1 | 20 | Good | Good |
| Example 2 | 40 | Fair | Fair |
| Example 3 | 10 | Fair | Good |
| Example 4 | 80 | Good | Good |
| Example 5 | 30 | Fair | Good |
| Comp. Ex. 1 | — | Bad | Bad |

It is understood from Table 1 that a fastening tab having an opening formed therein has better handleability than a fastening tab having two belts disposed thereon.

1. A fastening tab for a disposable diaper, the fastening tab comprising:
a base member having a first terminal end portion and a second terminal end portion for permanent attachment to the disposable diaper, and
a fastener adjacent the first terminal end portion of the base member;
wherein the base member is provided with at least one opening between the first and second terminal end portions or at least one notch on the seconds terminal end portion, which opening or notch extends through the entire base member.

2. A fastening tab for a disposable diaper according to claim 1, wherein at least a part of the base member is formed out of an elastic material.

3. A fastening tab for a disposable diaper according to claim 2, wherein the opening has a shape of a triangle, a rectangle, a square, a trapezoid, a circle, an ellipse, a race-track, a heart, or a crescent.

4. A fastening tab for a disposable diaper according to claim 2, wherein the elastic material is a film elastic laminated to a base layer.

5. A fastening tab for a disposable diaper according to claim 1, wherein the at least one opening or notch forms at least two belt-like portions.

6. A fastening tab for a disposable diaper according to claim 5, wherein each belt-like portion is at least 5 mm wide.

7. A fastening tab for a disposable diaper according to claim 5, wherein each belt-like portion is formed at least in part with elastic material.

8. A fastening tab for a disposable diaper according to claim 5, wherein each belt-like portion is associated with at least one discrete elastic member.

9. A fastening tab for a disposable diaper according to claim 8, wherein there are separate discrete elastic members associated with each belt-like portion.

10. A fastening tab for a disposable diaper according to claim 9, wherein the belt-like portions are created by the at least one notch.

11. A fastening tab for a disposable diaper according to claim 5, wherein the belt-like portions are created by the at least one notch.

12. A fastening tab for a disposable diaper according to claim 1, wherein the fastener adjacent the first terminal end portion is an attached fastener.

13. A fastening tab for a disposable diaper according to claim 12, wherein the fastening tab forms a side panel of the disposable diaper.

14. A fastening tab for a disposable diaper according to claim 1, wherein the fastening tab forms a side panel of the disposable diaper.

15. A fastening tab for a disposable diaper according to claim 2, wherein the elastic material is a discrete elastic member on an extensible inelastic base layer.

16. A fastening tab for a disposable diaper according to claim 5, wherein there are at least two discrete elastic members associated with the at least two belt-like portions.

17. A fastening tab for a disposable diaper according to claim 1, wherein the opening has a shape of a triangle, a rectangle, a square, a trapezoid, a circle, an ellipse, a race-track, a heart, or a crescent.

18. A disposable diaper comprising:
a main body comprising a top portion and a bottom portion; and
two side panel portions disposed on the main body adjacent one of the top portion or the bottom portion, wherein at least one side panel portion is provided with a fastening tab according to claim 1.
